(19) United States
(12) Patent Application Publication
(10) Pub. No.: US 2002/0007834 A1
(43) Pub. Date: Jan. 24, 2002

(54) Surgical drape

(76) Inventors: Patrick N. Trotter, Prattville, AL (US); Dorothy R. McMahan, Titus, AL (US)

Correspondence Address: John E. Toupal, 116 Concord Street, Framingham, MA, 01702, US

(21) Appl. No.: 09/899,605

(22) Filed: Jul. 5, 2001

Related U.S. Application Data
(60) Provisional application No. 60/219,416, filed on Jul. 20, 2000.

(51) Int. Cl. A61F 5/37

(52) U.S. Cl. 128/849

(57) ABSTRACT

A surgical drape including a flexible sheet having a top surface and an outer edge including distinct given and particular segments, the primary sheet defining a fenestration for accommodating a surgical procedure; a flexible first flap secured to the given segment and shaped and arranged to be foldable over the sheet into a position substantially covering the fenestration; and a flexible second flap secured to the particular segment and shaped and arranged to be foldable over the sheet into a position substantially covering the fenestration. The flaps are deployable to selectively reduce the size of the fenestration.

### BACKGROUND OF THE INVENTION

The present invention relates generally to surgical drapes and, more particularly, to surgical drapes with multiple components.

A various assortment of drapes have been proposed for surgical procedures, during which the drapes are used to cover the body of a patient and maintain a sterile barrier around the operative site while the procedure is performed through a fenestration in the drape. Typically, such drapes have been provided with fenestrations of a predetermined size for use in a particular surgical procedure depending upon the size and location of the surgical site. Accordingly, an excessive number of drapes are required, and drapes must sometimes be replaced or cut pursuant to related procedures. For example, procedures on children and adults require fenestrations of different size. Accordingly, it has been necessary for hospitals to maintain a stock of different drapes for performing the different procedures. Alternatively, the fenestration of a drape designed for a smaller surgical procedure must be cut to enlarge the fenestration for an expanded surgical procedure, which has been time consuming and inconvenient for the operating room team, as well as increasing the likelihood of contamination of the drape.

Disclosed in U.S. Pat. No. 5,778,891 is an improved surgical drape usable selectively for surgical procedures involving sites of various size. The disclosed surgical drape fails, however, to fully satisfy certain surgical needs.

The object of this invention, therefore, is to provide an improved surgical drape which can be used selectively for a wide variety of surgical procedures.

### SUMMARY OF THE INVENTION

The invention is a surgical drape including a flexible sheet having a top surface and an outer edge including distinct given and particular segments, the primary sheet defining a fenestration for accommodating a surgical procedure; a flexible first flap secured to the given segment and shaped and arranged to be foldable over the sheet into a position substantially covering the fenestration; and a flexible second flap secured to the particular segment and shaped and arranged to be foldable over the sheet into a position substantially covering the fenestration. The flaps are deployable to selectively reduce the size of the fenestration.

According to one feature of the invention, the first flap is foldable over the sheet in one direction and the second flap is foldable over the sheet in another direction transverse to the one direction. This feature facilitates selective, two-dimensional reduction in the size of the fenestration.

According to another feature of the invention, the top surface defines a fluid repellent border surrounding the fenestration, the first flap has a bottom surface including a fluid repellent outer boundary portion; and the second flap has a bottom surface including a fluid repellent outer boundary portion. The fluid repellent border and first and second flaps provide dimensional stability for the fenestration.

According to a further feature of the invention, the drape includes a first securement for fixing the first flap in a position covering one selective portion of the fenestration, and second securement for fixing the second flap in a position covering another selective portion of said fenestration. The securements retain the flaps in the selected positions.

According to an additional feature of the invention, the first securement is detachably secured to the bottom surface of the first flap, and the second securement is detachably secured to the bottom surface of the second flap. This feature facilitates securement of the flaps into selective positions.

According to still other features of the invention, the sheet is rectangular, the given and particular segments are adjacent edge portions of the outer edge and the fenestration is rectangular. These features facilitate selective size reduction of the fenestration.

The invention also encompasses a method for utilizing the surgical drape and including the steps of placing the drape on a patient; aligning the fenestration over a surgical site on the patient; and folding the first and second flaps into selective positions partially covering the fenestration. Such manipulation of the flaps establishes a desired reduced size for the fenestration.

### BRIEF DESCRIPTION OF THE DRAWINGS

These and other objects and features of the invention will become more apparent upon a perusal of the following description taken in conjunction with the accompanying drawings wherein:

FIG. 1 is a plan view of a surgical drape according to the invention;

FIG. 2 is a front view of the surgical drape shown in FIG. 1;

FIG. 3 is a left end view of the surgical drape shown in FIGS. 1 and 2;

FIG. 4 is a right end view of the surgical drape shown in FIGS. 1 and 2;

FIG. 5 is a plan view showing the surgical drape of FIG. 1 in a first selective deployment;

FIG. 6 is a plan view of the surgical drape shown in a second selective deployment;

FIG. 7 is a plan view of another surgical drape embodiment;

FIG. 8 is a left end view of the surgical drape shown in FIG. 7; and

FIG. 9 is a right end view of the surgical drape shown in FIG. 1.

### DESCRIPTION OF THE PREFERRED EMBODIMENTS

A surgical drape 11 includes a flexible sheet 12 having a top surface 13 and an outer edge 14 partially formed by a given segment 14 and an adjacent particular segment 15. Defined by the flexible sheet 12 is a fenestration 18 for accommodating a surgical procedure. A suitable fluid repellent strip 19 is secured, as by stitching, to the top surface 13 of the flexible sheet 12 so as to provide a fluid repellent border surrounding the fenestration 18. Preferably, the flexible sheet 12 and the fenestration 18 are rectangular as shown in FIG. 1.

Also included in the surgical drape 11 is a first flexible flap 21 secured to the given segment 15 of the edge 14 and a second flexible flap 22 secured to the particular segment 16 of the outer edge 14. Preferably, each of the first and second flaps 21, 22 has a surface area equal to that of the flexible sheet 12 as shown in FIG. 1. Secured, as by stitching, to an outer boundary portion of a bottom surface of the first flap 21 is a fluid resistant strip 26. Similarly secured to an outer boundary portion of a bottom surface 27 of the second flap 22 is a fluid repellent strip 28.

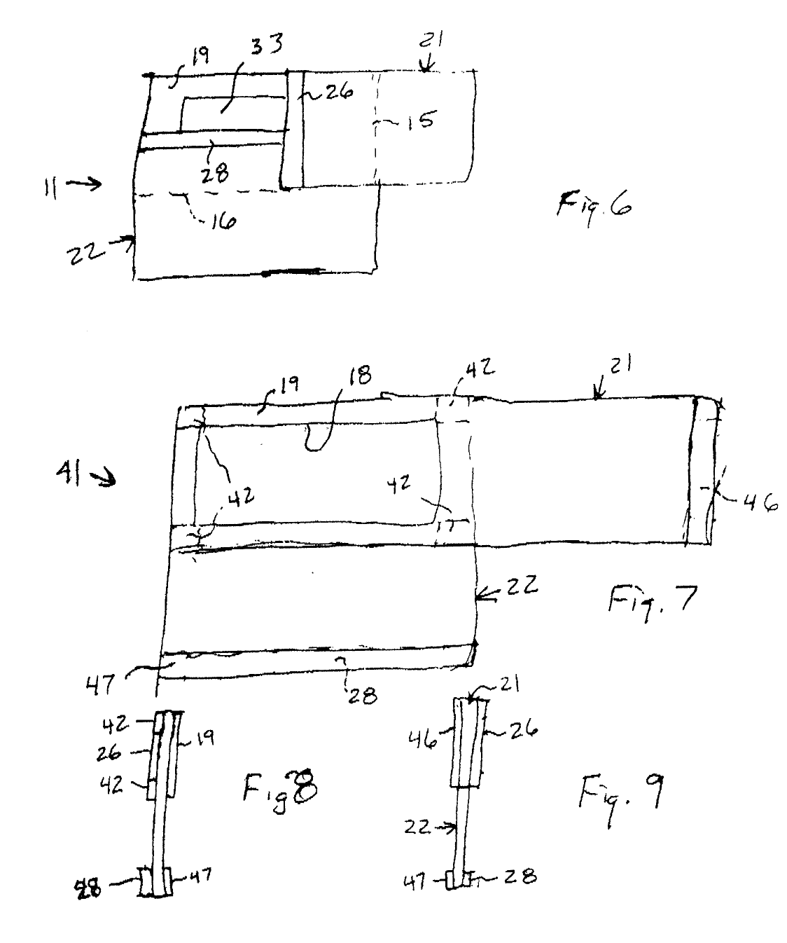

During use of the surgical drape 11, the second flap 22 can be folded over the top surface 13 of the flexible sheet 12 into a selective position partially covering the fenestration 18 as illustrated in FIG. 5. A resultant fenestration opening 31 of reduced size is bordered by a portion of the fluid repellent strip 19 of the flexible sheet 12 and the fluid repellent strip 28 of the second flap 22. The size of the fenestration 31 is selected to accommodate the particular surgical procedure being performed. As further depicted in FIG. 6, the size of the fenestration 18 can be further modified by folding the first flap 21 over the top surface 13 into a position selectively covering a portion of the reduced fenestration opening 31 established by the above described manipulation of the second flap 22. Surrounding the fenestration 33 are portions of the fluid repellent strips 19, 26 and 28. Preferably, the first and second flaps 21, 22 are folded over the flexible sheet 12 as shown in transversely related directions so as to provide two-dimensional reduction in the size of the original fenestration 18. That objective is accommodated by securement of the flaps 21, 22 to, respectively, the adjacent edge segments 15, 16 of the outer edge 14 of the rectangular flexible sheet 12.

Another surgical drape embodiment 41 is illustrated in FIGS. 7-9.

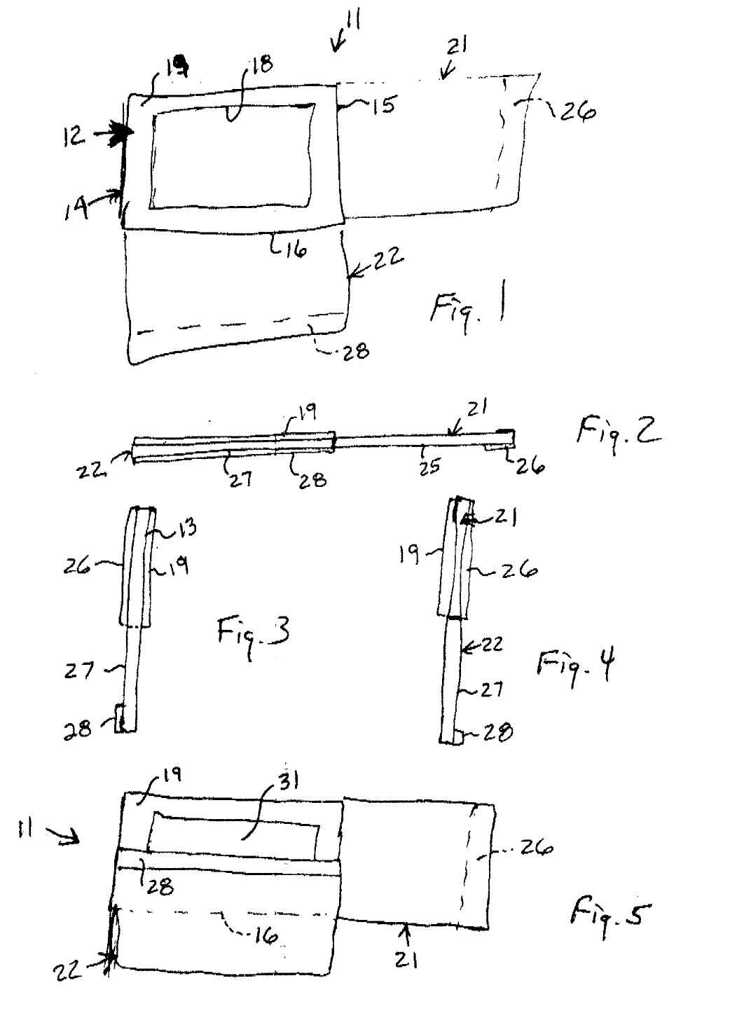

Components of the surgical drape 41 identical to those of the surgical drape 11 of FIGS. 1-6 bear identical reference numerals. Additional components of the surgical drape 41 include detachable adhesive tabs 42 attached to corner portions of a bottom surface 43 of the flexible sheet 12. In addition, detachable adhesive strips 46, 47 are secured to outer boundary portions of, respectively, the first and second flaps 21 and 22.

During use of the surgical drape 41 the adhesive tabs 42 are used to secure the sheet 12 over a patient's body portions surrounding the surgical procedure region. Subsequently, the adhesive strips 46, 47 function to secure, respectively, the first and second flaps 21, 22 into selective positions partially covering the fenestration 18 as described above.

Obviously, many modifications and variations of the present invention are possible in light of the above teachings. It is to be understood, therefore, that the invention can be practiced otherwise than as specifically described.

What is claimed is:

1. A surgical drape comprising:
a flexible sheet having a top surface and an outer edge including distinct given and particular segments, said primary sheet defining a fenestration for accommodating a surgical procedure;
a flexible first flap secured to said given segment and shaped and arranged to be foldable over said sheet into a position substantially covering said fenestration; and
a flexible second flap secured to said particular segment and shaped and arranged to be foldable over said sheet into a position substantially covering said fenestration.

2. A surgical drape according to claim 1 wherein said first flap is foldable over said sheet in one direction and said second flap is foldable over said sheet in another direction transverse to said one direction.

3. A surgical drape according to claim 2 wherein said top surface defines a fluid repellent border surrounding said fenestration, said first flap has a bottom surface including a fluid repellent outer boundary portion; and said second flap has a bottom surface including a fluid repellent outer boundary portion.

4. A surgical drape according to claim 2 including first securing means for fixing said first flap in a position covering one selective portion of said fenestration, and second securement means for fixing said second flap in a position covering another selective portion of said fenestration.

5. A surgical drape according to claim 4 wherein said first securement means is detachably secured to said first flap, and said second securement means is detachably secured to said second flap.

6. A surgical drape according to claim 5 wherein said first securement means is detachably secured to said bottom surface of said first flap, and said second securement means is detachably secured to said bottom surface of said second flap.

7. A surgical drape according to claim 2 wherein said sheet is rectangular, and said given and particular segments are adjacent edge portions of said outer edge.

8. A surgical drape according to claim 7 wherein said fenestration is rectangular.

9. A surgical drape according to claim 8 wherein said top surface defines a fluid repellent border surrounding said fenestration, said first flap has a bottom surface including a fluid repellent outer boundary portion; and said second flap has a bottom surface including a fluid repellent outer boundary portion.

10. A surgical drape according to claim 9 including first securing means for fixing said first flap in a position covering one selective portion of said fenestration, and second securement means for fixing said second flap in a position covering another selective portion of said fenestration.

11. A surgical drape according to claim 10 wherein said first securement means is detachably secured to said first flap, and said second securement means is detachably secured to said second flap.

12. A surgical drape according to claim 11 wherein said first securement means is detachably secured to said bottom surface of said first flap, and said second securement means is detachably secured to said bottom surface of said second flap.

13. A method for utilizing a surgical drape having a flexible sheet having a top surface and outer edge including distinct given and particular segments, the primary sheet defining a fenestration for accommodating a surgical procedure; a flexible first flap secured to the given segment and shaped and arranged to be foldable over the sheet into a position substantially covering the fenestration; and a flexible second flap secured to the particular segment and shaped and arranged to be foldable over the sheet into a position substantially covering the fenestration and comprising the steps of:
placing the drape on a patient;
aligning the fenestration over a surgical site on the patient;
folding the first flap in a selective position over the sheet with the outer edge thereof intersecting the fenestration so as to reduce the size thereof.

14. A method according to claim 13 including the step of folding the second flap in a selective position over the sheet with the outer edge thereof intersecting the fenestration so as to further reduce the size thereof.

15. A method according to claim 14 wherein said first flap is foldable over said sheet in one direction and said second flap is foldable over said sheet in another direction transverse to said one direction.

16. A method according to claim 15 wherein said top surface defines a fluid repellent border surrounding said fenestration, said first flap has a bottom surface including a fluid repellent outer boundary portion; and said second flap has a bottom surface including a fluid repellent outer boundary portion.

17. A method according to claim 16 wherein said drape includes first securing means for fixing said first flap in a position covering one selective portion of said fenestration, and second securement means for fixing said second flap in a position covering another selective portion of said fenestration.

18. A method according to claim 17 wherein said first securement means is detachably secured to said first flap, and said second securement means is detachably secured to said second flap; and including the steps of securing the first and second securing means to the top surface of the sheet.

19. A method according to claim 18 wherein said first securement means is detachably secured to said bottom surface of said first flap, and said second securement means is detachably secured to said bottom surface of said second flap.

20. A method according to claim 19 wherein said sheet is rectangular, and said given and particular segments are adjacent edge portions of said outer edge.
